(19) United States
(12) Patent Application Publication
(10) Pub. No.: US 2018/0116789 A1
(43) Pub. Date: May 3, 2018

(54) Speech Prosthesis Employing Beta Peak Detection

(71) Applicant: Philip R. Kennedy, Duluth, GA (US)

(72) Inventor: Philip R. Kennedy, Duluth, GA (US)

(73) Assignee: Neural Signals, Inc., Duluth, GA (US)

(21) Appl. No.: 15/800,589

(22) Filed: Nov. 1, 2017

Related U.S. Application Data
(60) Provisional application No. 62/415,812, filed on Nov. 1, 2016.

(51) Int. Cl. A61F 2/20; G10L 13/047; A61B 5/0478; A61B 5/00; A61B 5/048

(52) U.S. Cl. CPC A61F 2/20; G10L 13/047; A61B 5/0478; A61B 5/4851; A61B 5/0006; A61F 2002/482; A61B 5/7246; A61B 5/7257; A61B 5/6814; A61F 2002/206; A61B 5/048

(57) ABSTRACT

A speech synthesis device detects beta peak firings corresponding to intended vocalizations by a subject having a scalp, a mastoid process and a speech motor cortex. A EEG electrode and a negative electrode are disposed on the scalp of the subject adjacent to the speech motor cortex. A transmitter is electrically coupled to the electrodes, and is configured to transmit wirelessly electronic representations of the neural potentials detected by the electrodes. A remote unit receives the electronic representations of the neural potentials from the transmitter. The remote unit is programmed to: detect beta peaks firings in the neural potentials; correlate the beta peaks firings with beta peaks associated with phonemes, words and phrases; and generate audible representations of the phonemes, words and phrases.

### CROSS-REFERENCE TO RELATED APPLICATION(S)

This application claims the benefit of U.S. Provisional Patent Application Ser. No. 62/415,812, filed Nov. 1, 2016, the entirety of which is hereby incorporated herein by reference.

### BACKGROUND OF THE INVENTION

### 1. Field of the Invention

The present invention relates to medical systems and, more specifically, to a system for generating speech and other sounds based on neural impulses.

### 2. Description of the Related Art

Locked-in syndrome (LIS) is a clinical condition in which subjects suffer from complete paralysis and cannot speak but are awake and cognitively intact. This syndrome results from pontine ischemic or hemorrhagic strokes, amyotrophic lateral sclerosis (ALS), and other etiologies. It has been a long-term goal for many researchers to provide these subjects with a means of communication. Currently, assistive communication for locked-in individuals can be achieved via various devices such as external or EMG switches, EEG, ECOG, or by using implanted electrodes within the brain. The external noninvasive methods to produce speech output are inherently slow, with speech sounds being produced from a computer speaker after the subject has slowly spelled out what he/she wants to say. Decoding of neuronal activity from the cortical speech area is more likely to provide a more natural communication rate, perhaps approaching conversational speed. Efforts to decode speech phonemes from a locked-in subject using single unit activity have been partially successful to date. A specific roadblock remains the real-time detection of the onset of attempted vocalization.

In certain subjects, communication may be effected by sensing eye movements. In one communication method, the movement of the subject's eye is correlated to a table of letters displayed on a computer screen and the subject spells out words by looking at the letters that form the words that the subject wants to communicate. The result may be fed into a speech generator, which makes sounds corresponding to the words indicated by the subject. Alternately, inputs other than eye movement, such as motor-neural impulses, may be used to facilitate communications. In such systems, the input may control a cursor that moves over letters or icons on a computer screen and if the cursor rests on a letter for a sufficient amount of time, then the letter is added to a string of letters that eventually forms a word.

Such systems are limited in that they take a considerable amount of time to generate even simple words and they require the subject to expend extra mental effort in determining which letters are needed and the location of the letters on the table.

One system uses neural impulses sensed by electrodes implanted in a patient's brain to generate phonemes. This system trains the patient to think of a word that the patient wants to say and then recognizes neural potentials sensed by the electrodes. The pattern of the neural potentials is then correlated to a specific phoneme. The correlated phoneme is then generated by a computer. This system is highly invasive as it requires implantation of electrodes into the patient's brain.

Therefore, there is a need for a non-invasive system for detecting neural impulses corresponding to sounds that a patient desires to make.

### SUMMARY OF THE INVENTION

The disadvantages of the prior art are overcome by the present invention which, in one aspect, is a speech synthesis device for detecting beta peak firings corresponding to intended vocalizations by a subject having a scalp, a mastoid process and a speech motor cortex. At least one positive EEG electrode and at least one negative electrode are disposed on the scalp of the subject adjacent to the speech motor cortex. A transmitter is electrically coupled to the at least one positive EEG electrode and the at least one negative EEG electrode, and is configured to transmit wirelessly electronic representations of the neural potentials detected by the at least one positive EEG electrode and the at least one negative electrode. A remote unit includes receiver circuitry that receives the electronic representations of the neural potentials from the transmitter. The remote unit is programmed to: detect beta peaks firings in the neural potentials; correlate the beta peaks firings with beta peaks associated with phonemes, words and phrases; and generate audible representations of the phonemes, words and phrases.

In another aspect, the invention is a method for detecting intended vocalizations by a subject having a scalp, a speech motor cortex and a mastoid process, in which the subject is instructed to attempt to make a training vocalization. At least one positive EEG electrode and at least one negative EEG electrode is applied to a preferred electrode placement site so as to detect neural signals corresponding to intended vocalizations. Beta peak firings are detected in the electronic representations of the neural signals. The beta peak firings are correlated to beta peak firings associated with phonemes, words and phrases. Audible sounds corresponding to the phonemes, words and phrases are generated.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a schematic diagram of one embodiment of a speech prosthesis.

FIG. 2 is a schematic diagram of a multi-electrode embodiment of a speech prosthesis in use.

FIG. 3 is a graph showing power spectral density vs. frequency of detected neural potentials.

FIG. 4 is a flowchart showing one embodiment of a method of using a speech prosthesis.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

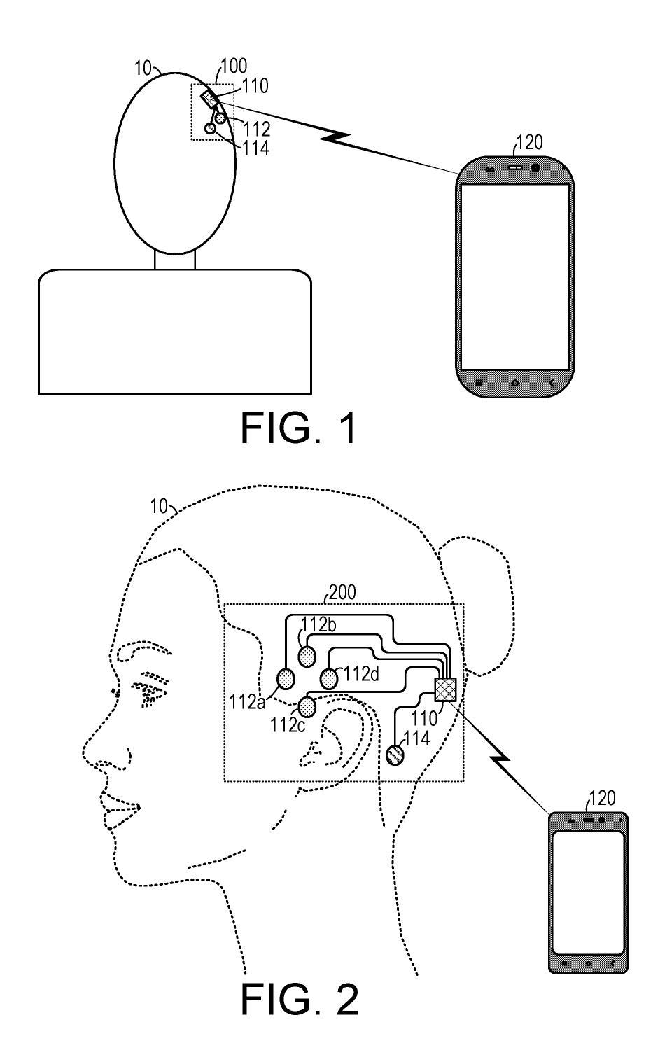

As shown in FIG. 1, one embodiment of a speech prosthesis includes a local unit 100, that is applied to the scalp of a subject 10, and a remote unit 120. The local unit includes a positive EEG electrode 112 and a negative EEG electrode 114 that are coupled to a transmitter 110. The transmitter 110 can include one of the many short distance technologies known to the art, including wireless technologies such as BLUETOOTH, etc. The transmitter 110 transmits neural impulse data from the electrodes 112 and 114 to a remote unit 120, which could include a cellular telephone, a laptop computer on one of many other computing devices known to the art.

In use, the subject 10 is trained to mentally attempt to say phrases, words or phonemes and electronic representations of the neural impulses that result for the attempts are sensed by the electrodes 112 and 114, and are transmitted to the remote unit 120 by the transmitter 110. The remote unit 120 digitizes the incoming signal and performs a fast Fourier transform on it thereby generating a frequency domain representation of the signal. In the frequency domain, beta peak firings above a predetermined threshold are detected and the values of the peak firings are stored in association with the phrases, words or phonemes that the subject 10 was attempting to say. After training, the subject 10 may attempt again to say the phrases, words or phonemes for which the subject 10 previously trained. The resulting beta peak firings are then correlated to the stored beta peak firings and corresponding audible sounds corresponding to the detected phrases, words or phonemes are generated by the remote unit 120. In alternate embodiments, the detected phrases, words or phonemes can also be displayed in the form of text or used as controls for other systems (such as turning on lights, fans, etc). In one experimental embodiment, it has been found that the most effective detections of beta peak firings are found in the following frequency ranges: 12 Hz to 20 Hz; 20 Hz to 30 Hz; and 12 Hz to 30 Hz.

As shown in FIG. 2, more than one positive electrode may be employed. In the figure shown, four positive electrodes (electrodes 112a-112d) are employed. Only one negative electrode 114 is necessary to provide a reference. The positive electrodes 112a-112d are placed on the scalp adjacent to the speech cortex of the subject 10. While a functional MM may be performed on the subject to determine optimal placement of the electrodes 112a-112d, it is generally known that the speech cortex is located slightly in front of and above the ear. Subjects who are right handed tend to exhibit stronger speech-related neural impulses on the left side of the head, whereas subjects who are left handed tend to exhibit stronger speech-related neural impulses on the right side of the head.

The negative electrode 114 is typically placed adjacent to the mastoid process. This is done because there are no muscles in the area of the scalp adjacent to the mastoid process and, therefore, placement of the negative electrode 114 there eliminates EMG artifacts in the resulting neural impulse signals.

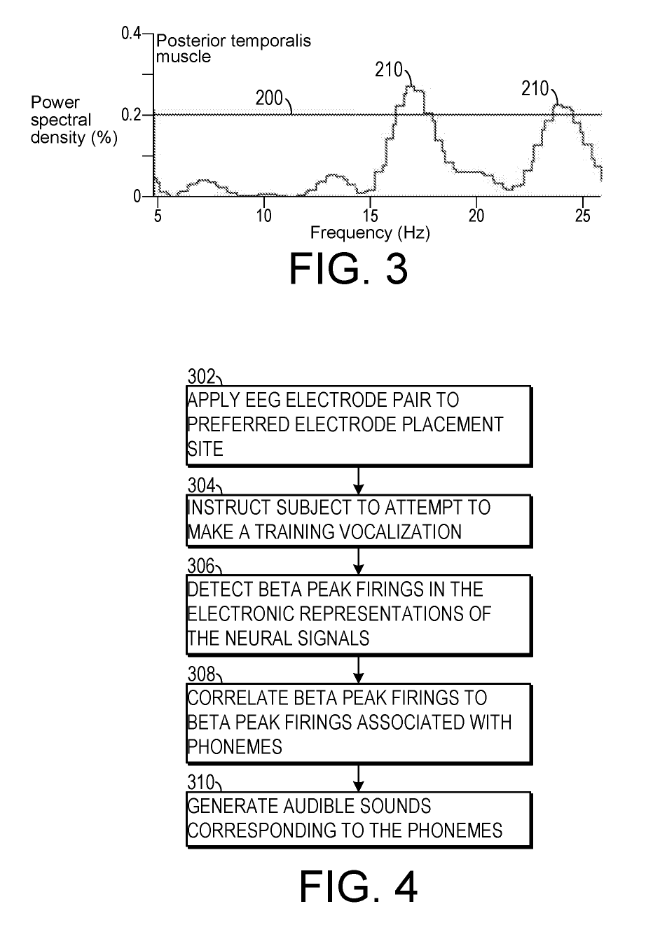

A graph showing a representative digitized frequency domain signal is shown in FIG. 3. This graph shows power spectral density of the signal as a function of frequency. The beta peaks 210 are at signal values that extend above a predetermined threshold 200.

As shown in FIG. 4, in one embodiment of a method of using the invention, the electrodes are applied to the placement site so as to detect neural signals corresponding to intended vocalizations 302 and the subject is instructed to attempt to make a training vocalization 304. Beta peak firings are detected 306 and the beta peak firings are correlated with stored beta peak firings 308. The remote unit then generates audible sounds corresponding to the phonemes, words and phrases 310.

In one experimental embodiment, it was discovered that data recorded from the motor speech area of aphasic locked-in and awake speaking subjects has revealed a consistent lower beta peak frequency of 12 to 20 Hz. This beta peak was shown to be present at the onset of covert speech. Studies in the speaking subject revealed that the beta peaks were also present at the onset, offset and inflection point in words and phrases. This raises the possibility of developing a speech prosthesis using only external recording from the scalp, thus avoiding implantation of electrodes within the brain or on its surface.

Such a speech prosthesis uses the pattern of beta peak firings to detect 10 or more short words. One embodiment of a system consists of wireless recording of the beta peaks and their transmission to a cell phone app that would detect the beta peaks and their firing patterns and output the corresponding words through the phone speakers.

In one embodiment, external recordings of beta peaks are sensed from EEG electrodes that are held in position using EEG paste (such as, EC2, Natus Manufacturing, Gort, Co. Galway, Ireland). The active electrode (red wire) is positioned one inch above the negative electrode (green wire) in the direction of the vertex as shown in the subject (who, in this case, is right handed). The common electrode is placed on the right mastoid bone.

In the experimental embodiment, identification of the site for electrodes was achieved using functional MRI with the mute subject making silent (not imagined) vocalizations on an object naming task while in the MRI scanner. In the experimental embodiment, a speaking subject made repeated movements of his tongue, cheeks and jaw, and locations of vascular activity in the fMRI were unique. In addition the electrode site was narrowed even further to the lateral aspect of the premotor face area that extends from the Sylvian fissure to 1″ medially. Thus, the external electrodes are placed over this area on the scalp.

Recording and Analysis of Data:

The electrode wires were fed into a CWE amplifier (such as, BMA 200, CWE, Ardmore, Pa., USA). Gain was set at 500, with filters set to 1 Hz to 10 KHz. The output was fed into Neuralynx's Cheetah archiving software. Speech output is recorded with the microphone set at a fixed distance from the subject's mouth and fed into the Cheetah software. During acquisition, a circuit was closed using a button push with the subject's left hand (to avoid contaminating the beta peak due to hand movement, while the right hand remained quiescent). The archived data were analyzed for beta peaks using Neuroexplorer software (version 3.259, NEX Nex Technologies, Madison, Ala., USA). The frequency range was restricted between 12 to 20 Hz The data are analyzed in 150, 200, 250, 300, 350, 400, 450 and 500 ms time bins. The criteria for choosing acceptable responses include peaks that lie in the 14 and 15 Hz region using any time bin, and whose baseline is no higher than 20% of the total peak amplitude using percentage of power spectral density analysis.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A speech synthesis device for detecting beta peak firings corresponding to intended vocalizations by a subject having a scalp, a mastoid process and a speech motor cortex, comprising:
(a) at least one positive EEG electrode and at least one negative electrode disposed on the scalp of the subject adjacent to the speech motor cortex;
(b) a transmitter, electrically coupled to the at least one positive EEG electrode and the at least one negative EEG electrode, that is configured to transmit wirelessly electronic representations of the neural potentials detected by the at least one positive EEG electrode and the at least one negative electrode; and
(c) a remote unit that includes receiver circuitry that receives the electronic representations of the neural potentials from the transmitter and that is programmed to:
(i) detect beta peaks firings in the neural potentials;
(ii) correlate the beta peaks firings with beta peaks associated with phonemes, words and phrases; and
(iii) generate audible representations of the phonemes, words and phrases.

2. The speech synthesis device of claim 1, wherein the remote unit comprises a cellular telephone.

3. The speech synthesis device of claim 1, wherein the remote unit is further programmed to perform a fast Fourier transform on data received from the at least one positive EEG electrode and at least one negative EEG electrode prior to detecting beta peak firings.

4. The speech synthesis device of claim 1, further comprising at least one second positive EEG electrode.

5. The speech synthesis device of claim 1, wherein the positive electrode is disposed apart from the negative electrode by a predetermined distance.

6. The speech synthesis device of claim 1, wherein the at least one positive electrode is disposed adjacent to the speech motor cortex.

7. The speech synthesis device of claim 1, wherein the negative electrode is disposed adjacent to the mastoid process.

8. The speech synthesis device of claim 1, wherein the beta peak firings are found at frequency domain ranges selected from a list consisting of: 12 Hz to 20 Hz; 20 Hz to 30 Hz; and 12 Hz to 30 Hz.

9. A method for detecting intended vocalizations by a subject having a scalp, a speech motor cortex and a mastoid process, comprising the steps of:
(a) applying at least one positive EEG electrode and at least one negative EEG electrode to a preferred electrode placement site so as to detect neural signals corresponding to intended vocalizations;
(b) instructing the subject to attempt to make a training vocalization;
(c) detecting beta peak firings in the electronic representations of the neural signals;
(d) correlating the beta peak firings to beta peak firings associated with phonemes, words and phrases; and
(e) generating audible sounds corresponding to the phonemes, words and phrases.

10. The method of claim 9, further comprising the step of performing a fast Fourier transform on data received from the at least one positive EEG electrode and at least one negative EEG electrode prior to the step of detecting beta peak firings.

11. The method of claim 9, further comprising the step of wirelessly transmitting electronic representations of the neural signals to a remote unit, wherein the steps of detecting beta peak firings, correlating beta peak firings and generating audible sounds are performed by the remote unit.

12. The method of claim 11, wherein the remote unit comprises a cellular telephone.

13. The method of claim 9, further comprising the step of applying at least one second positive EEG electrode to the preferred electrode placement site.

14. The method of claim 9, wherein the step of applying at least one positive EEG electrode and at least one negative EEG electrode to a preferred electrode placement site, comprises the steps of applying the at least one positive electrode to the scalp adjacent to the speech motor cortex and applying the at least one negative electrode to the scalp adjacent to the mastoid process.

15. The method of claim 9, further comprising the step of performing a functional MRI on the subject as the subject attempts to make the training vocalization, thereby detecting a preferred electrode placement site.

16. The method of claim 9, wherein the step of detecting the beta peak firings occurs at frequency domain ranges selected from a list consisting of: 12 Hz to 20 Hz; 20 Hz to 30 Hz; and 12 Hz to 30 Hz.
